(19) United States
(12) Patent Application Publication
(10) Pub. No.: US 2008/0013814 A1
(43) Pub. Date: Jan. 17, 2008

(54) Pharmacokinetic Image Registration

(75) Inventor: Ingwer Carlsen, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/568,548

(22) PCT Filed: Apr. 26, 2005
(86) PCT No.: PCT/IB05/51349

§ 371 Date: Nov. 1, 2006

(30) Foreign Application Priority Data
May 6, 2004 (EP) 04101958.9

(51) Int. Cl. A61B 5/05

(52) U.S. Cl. 382/131

(57) ABSTRACT

Image registration very often used to be a tedious task which had to be performed manually. According to an exemplary embodiment of the present invention, a registration of image time series is performed on the basis of a pharmacokinetic model in which alternative translation sequences of a region of interest are compared to each other on the basis of the pharmacokinetic model and the best translation vector sequence is used for image registration. Advantageously, this may allow for an effective compensation of organ movement, even if there is no or only little anatomical contrast.

The present invention relates to the field of digital imaging, for example in the field of medical imaging. In particular, the present invention relates to a method of registering time series of images comprising at least a first image and a second image, to an image processing device, to scanner systems and to a computer program for registering a first image and a second image.

When two images of the same object have to be taken from different projections or at different points of time, or during different moving stages of the object of interest, it may be highly desirable to register the images.

Registering images means the integration of their geometric properties to a common system of reference. Normally, this is done by warping the images until corresponding anatomical gray values structures match with respect to some similarity measure such as a cross-correlation, a mutual information, etc. The registration of nuclear medical images becomes more difficult with growing specificity of the tracers. The more specific a tracer, the less its general tissue up-take and the less, accordingly, the anatomical background contrast it produces (problem of lost anatomical contrast), turning conventional gray value based registration unreliable.

It is an object of the present invention to provide for an improved image registration.

According to an exemplary embodiment of the present invention as set forth in claim 1, the above object may be solved by a method of registering a time series of images, the time series comprising a first image and a second image, wherein a first region of interest is selected in the first image and a second region of interest is selected in the second image. Furthermore, a first translation of the first region of interest to the second region of interest is determined on the basis of a pharmacokinetic model. The first image and the second image are registered on the basis of the first translation, wherein the first region of interest corresponds to the second region of interest.

For example, according to this exemplary embodiment of the present invention, a local registration of a time series of images comprising at least two images is performed by determining a translation of a specific region of interest, such as a piece of cancerous tissue, over a specific period of time. Advantageously, according to this exemplary embodiment of the present invention, the translation is determined on the basis of a pharmacokinetic model, therefore allowing for a tracking of the region of interest for images with little or even no anatomical contrast, as is frequently the case when highly specific tracers are used in nuclear/medical imaging.

According to another exemplary embodiment of the present invention as set forth in claim 2, the first translation is identified by determining a first pharmacokinetic parameter on the basis of a second translation from the first region of interest to a third region of interest selected in the second image and on the basis of the pharmacokinetic model. Furthermore, a second pharmacokinetic parameter is determined on the basis of a third translation from the first region of interest to a fourth region of interest selected in the second image and on the basis of the pharmacokinetic model. The third translation is a variation of the second translation and the third and fourth regions of interest correspond to the first region of interest. Furthermore, a quality determination of the first compartment parameter and the second compartment parameter is performed, resulting in a first quality value and a second quality value. Then, according to this exemplary embodiment of the present invention, the first and second quality values are compared and it is determined, which one of the first and second quality values is a better quality value. A translation is selected from the second translation and the third translation. The selected translation corresponds to the better quality value, wherein the selected translation is the first translation.

Advantageously, according to this exemplary embodiment of the present invention, different (alternative) translations of an object of interest are compared by estimating respective pharmacokinetic parameters on the basis of the different translations by applying a pharmacokinetic model. The pharmacokinetic parameters are then qualified, resulting in respective quality values. The “better” translation (which is the translation corresponding to the “better” quality value) is then used for image registration.

Advantageously, this may allow for an improved image registration.

Another exemplary embodiment of the present invention is set forth in claim 3, in which the quality determination is performed on the basis of at least one of a determination of a statistical quality measure on the basis of at least one of the first pharmacokinetic parameter estimate and the second pharmacokinetic parameter estimate, a library of pharmacokinetic parameters, and a consistency of the first pharmacokinetic parameter estimate and the second pharmacokinetic parameter estimate.

Advantageously, this may allow for a fast, efficient or even automatic quality determination.

According to another exemplary embodiment of the present invention as set forth in claim 4, at least one of the first and second regions of interest and the pharmacokinetic model are interactively selected from a predefined set of options. Therefore, according to this exemplary embodiment of the present invention, a user may choose a candidate lesion and the pharmacokinetic model which is to be applied, such as a pharmacokinetic model, shortly after or even during image acquisition.

Advantageously, this may allow for a fast and user-friendly interactive image registration.

According to another exemplary embodiment of the present invention as set forth in claim 5, the method of image registration is iteratively repeated until at least one of the first quality value and the second quality value exceeds a preset threshold value.

According to another exemplary embodiment of the present invention as set forth in claim 6, the method is applied in medical imaging on one of CT data sets, MRI data sets, PET data sets, SPECT data sets and ultrasound imaging data sets.

According to another exemplary embodiment of the present invention as set forth in claim 7, an image processing device for registering a first image and a second image is provided, comprising a memory for storing a multi-dimensional data set comprising the first image and the second image and an image processor adapted for performing the following operation: loading the multi-dimensional data set; selecting a first region of interest in the first image; selecting a second region of interest in the second image; determining a first translation from the first region of interest to the second region of interest on the basis of a pharmacokinetic model and registering the first image and the second image on the basis of the first translation. The first region of interest corresponds to the second region of interest.

Advantageously, the image processing device according to this exemplary embodiment of the present invention may allow for an improved image registration speed and a high registration accuracy.

The present invention also relates to scanner systems comprising a memory for storing a multi-dimensional data set comprising a first image and a second image and an image processor adapted for performing a registration of the first image and the second image. According to an aspect of the present invention, the scanner system is one of a CT scanner system, an MRI scanner system, a PET scanner system, an SPECT scanner system, and an ultrasound imaging system. The scanner systems according to the present invention are set forth in claims 8 and 9.

Advantageously, this may allow for an improved image registration of a time series of images acquired by a scanner system according to the present invention.

The present invention also relates to computer programs, which may, for example, be executed on a processor, such as an image processor. Such computer programs may, for example, be part of a CT scanner system, an MRI scanner system, a PET scanner system, a SPECT scanner system or an ultrasound system. The computer programs according to an exemplary embodiment of the present invention are set forth in claim 10. These computer programs may be preferably loaded into working memories of image processors. The image processors are thus equipped to carry out exemplary embodiments of the present invention. The computer programs may be stored on a computer readable medium, such as a CD-ROM. The computer programs may also be presented over a network such as the WorldWideWeb and may be downloaded into the working memory of an image processor from such networks. Computer programs according to this exemplary embodiment of the present invention may be written in any suitable programming language, such as C++.

It may be seen as the gist of an exemplary embodiment of the present invention that a registration of image time series is performed on the basis of a pharmacokinetic model such as a compartment model in which alternative translation sequences of a region of interest (which moves during image acquisition, due to, for example, patient motion) are compared to each other on the basis of the pharmacokinetic model and the best translation vector is used for image registration. Advantageously, this may allow for an effective compensation of organ movement, even if there is no or only little anatomical contrast. Advantageously, according to an exemplary embodiment of the present invention, a threshold value may be introduced and the procedure of comparing different possible translation vectors may be iteratively repeated until a quality value corresponding to a quality of a respective translation vector exceeds the threshold value.

These and other aspects of the present invention will become apparent from and elucidated with reference to the embodiments described hereinafter.

Exemplary embodiments of the present invention will be described in the following, with reference to the following drawings:

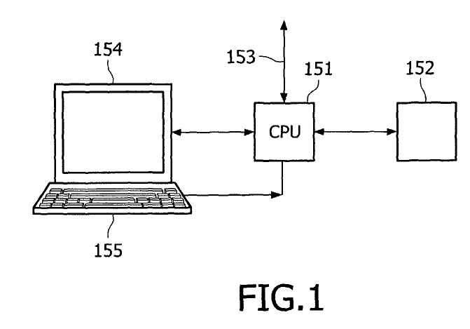

FIG. 1 shows an exemplary embodiment of an image processing device according to the present invention, for executing an exemplary embodiment of a method in accordance with the present invention.

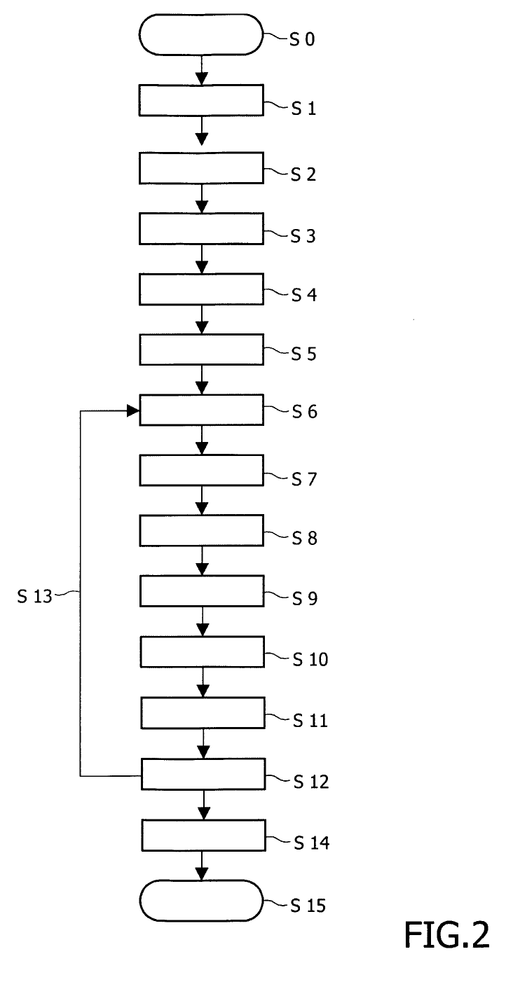

FIG. 2 shows a flow-chart of an exemplary embodiment of a method of registering images according to the present invention.

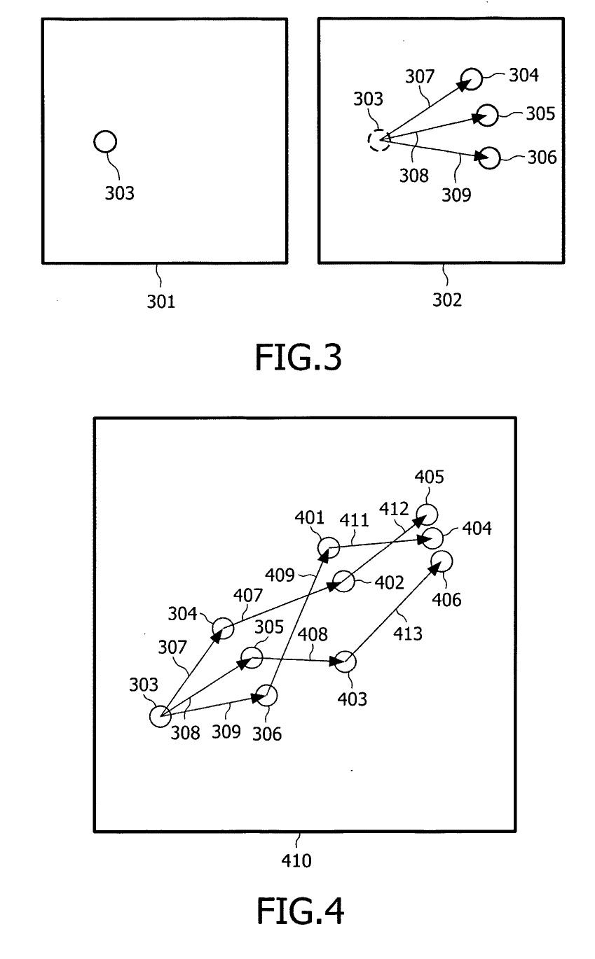

FIG. 3 shows a schematic representation of images and operations performed for registering the image according to an exemplary embodiment of the present invention.

FIG. 4 shows a schematic representation of an operation performed for registering images according to another exemplary embodiment of the present invention.

FIG. 1 shows an exemplary embodiment of an image processing device according to the present invention for executing an exemplary embodiment of a method in accordance with the present invention. The image processing device depicted in FIG. 1 comprises a central processing unit (CPU), an image processor 151 connected to a memory 152 for storing a multi-dimensional data set comprising images depicting an object of interest, such as an inner organ comprising a region of interest (for example cancerous tissue). The image processor 151 may be connected to a plurality of input/output network devices, such as an MR device or a CT device. The image processor is furthermore connected to a display device 154, for, for example, a computer for displaying information or an image computed or adapted in the image processor 151. An operator may interact with the image processor 151 via a keyboard 155 and/or other output or input devices, such as a computer mouse, which are not depicted in FIG. 1. Furthermore, via the bus system 153 it is also possible to connect the image processing and control processor 151 to, for example, a motion monitor which monitors a motion of the object of interest. In case, for example, a lung of a patient is imaged, the motion sensor may be an exhalation sensor. In case the heart is imaged, the motion sensor may be an electrocardiogram.

FIG. 2 shows a flow-chart of an exemplary embodiment of a method of image registration according to an exemplary embodiment of the present invention. The method starts at step S0, after which an acquisition of a multi-dimensional data set is performed in step S1, for example, by means of a polychromatic source of electro-magnetic radiation generating a polychromatic beam and by means of a radiation detector detecting the polychromatic beam, which is the case in, for example, CT imaging.

It should be noted that the acquired multi-dimensional data set may be a time series of 2-dimensional data sets or 3-dimensional data sets. Furthermore, the data set may comprise additional information, e.g. information about a periodic motion, for example by means of electro-cardiogram data acquired during image acquisition.

The electro-cardiogram data may be used for performing a pre-motion compensation (before image registration is performed) on the basis of the heart beat rate (if, for example, the heart of a patient is imaged). Furthermore, if, for example, a lung of a patient is imaged, the multi-dimensional data set may comprise data measured by an exhalation sensor.

The method depicted in FIG. 2 is an exemplary embodiment of a model-based mechanism for the local registration of a time series of nuclear-medical images on the basis of a so-called pharmacokinetic model (which is a compartment model).

A pharmocokinetic model describes the flow of a specific pharmaceutical substance in a human or animal body from the administration to the final excretion. A compartment model is a special mathematical representation of such a pharmocokinetic model. It simplifies the flow of the pharmaceutical substance through the body on a heuristical level to the exchange of this substance between a certain number of compartments or reservoirs that are coupled by a number of exchange rates describing the net in- and out-flow of the substance per compartment. Balancing the flow rates between the compartments allows to described the time dependent content of the substance in each of the compartments in terms of the exchange rates, the k-parameters. Such pharmacokinetic models or compartment models are well known in the art and therefore will not be described in greater detail.

For detecting the time dependency of tracer up-take, which is an important diagnostic tool for determining the malignancy of lesions and their response to therapy, time series of nuclear-medical images are acquired and the time variation of specific up-take values by the candidate lesion is measured. The pharmacokinetic model describes the time dependency of the specific up-take values in terms of the characteristic parameters k₁, . . . , kₙ modeling the tracer flow between so-called compartments (compartment models).

In order to obtain accurate specific up-take values and derive estimates of the characteristic k-parameters, the candidate lesion has to be tracked throughout the time series, to compensate for patient and organ motion to make sure that specific up-take values are obtained from the same anatomical domain throughout the course of time.

In step S2, a selection of a time series of image slices or volume data (3-dimensional images) from the multi-dimensional data set is performed. After that, in step S3, a first region of interest, such as a candidate lesion, is selected in a first image of the data set. Then, in step S4, a second region of interest in a second image of the data set is selected. The second image may be an image following the first image with respect to time (meaning that it is acquired after the first image is acquired). The second region of interest corresponds to the first region of interest such that it corresponds to the same candidate lesion but at a different point in time and, due to, for example, organ movement, at a different point in space.

After that, in step S5, a first translation is determined, which describes the translation of the first region of interest in the first image to the second region of interest in the second image. Furthermore, in step S6, an alternative second translation is determined, describing a translation from the first region of interest in the first image to a third region of interest in the second image, wherein the third region of interest is slightly shifted with respect to the second region of interest. In step S7, further images (of later point in time) may be selected from the multi-dimensional data set (such as a third image and a fourth image), and a translation of the candidate lesion is tracked from image to image, resulting in a first sequence of translation vectors, describing the motion of the candidate lesion from the first image to the second image, to the third image and to the fourth image, and in a second sequence of translation vectors, which is a slight variation of the first translation vector sequence, describing an alternative track of the region of interest (candidate lesion) from the first image to the second image, to the third image and to the fourth image (step S8). Therefore, in case of a time series of four images, the first and second translation vectors sequence each comprise 3 translation vectors each of which have a dimensionality of two or three depending on the dimensionality of the input images. In general, a time series of n images results in a translation vector sequence of (n-1) two- or three-dimensional translation vectors.

After that, in step S9, a first compartment vector K₁ (which is a vector (k_{1,1}, k_{1,2}, k_{1,3}, k_{1,4})) comprising all k-parameters of the compartment model of interest is determined on the basis of the first translation vector sequence describing the translation of the lesion from the first region of interest in the first image, to, for example, a fourth region of interest in the fourth image (via the second region of interest in the second image and the third region of interest in the third image). Furthermore, this compartment vector K₁ is determined on the basis of the pharmacokinetic model (which is a compartment model).

Furthermore, a second compartment vector K₂=(k_{2,1}, k_{2,2}, k_{2,3}, k_{2,4}) is obtained on the basis of the second translation vector sequence describing the translation of the first region of interest in the first image to a fifth region of interest in the fourth image, wherein the fifth region of interest in the fourth image is slightly shifted with respect to the fourth region of interest in the fourth image (described by the first translation vector). Thus, the second translation vector sequence is a variation of the first translation vector sequence and describes a second track of the lesion through the time series of images.

After determining the compartment parameters on the basis of a compartment model, a quality determination of the first compartment vector and the second compartment vector is performed, resulting in a corresponding first quality value and a corresponding second quality value (step S10).

The quality determination which results in the first and second quality values may be performed on the basis of a statistical quality of the k-estimates, for example their statistical variance, or on the basis of a given library of possible sets of k-values for the lesion and anatomy imaged, which results in a distance in feature space from the closest set of matching k-values (k-vector). This distance in feature space may be used as the quality value. Furthermore, according to an exemplary embodiment of the present invention, the quality determination may be performed on the basis of a consistency of the k-estimates obtained from registering the image series forwards (I₁→I₂→I₃→I₄)and backwards (I₄→I₃→I₂→I₁) in time.

However, it should be noted that other choices of quality determination procedures are possible.

After that, in step S11, the first and second quality values are compared to each other and it is determined which of them is the “better” quality value. The “better” quality value is further processed. In step S12 it is determined whether the “better” quality value exceeds a preset threshold value or, in other words, whether the “better” quality value meets a certain threshold criteria. If, in step S12, it is determined that the “better” quality value does not meet the preset threshold criteria, which may have been manually set by a user or automatically from the software side, the method jumps back to step S6, in which a further alternative translation vector sequence is determined. This alternative further translation vector sequence is again a further variation of the first translation vector sequence, or it may be a variation of the second translation vector sequence, depending on which of the two translation vector sequences resulted in the “better” quality value.

If, in step S12, it is determined that the threshold criterion is met, the method continues with step S14, in which a registration of the four images is performed. After registration, the method ends in step S15.

It should be noted that, according to an exemplary embodiment of the present invention, the regions of interest may be selected from a predefined set of options. Furthermore, the compartment model may be selected from a predefined set of compartment models. Advantageously, the selection may be performed interactively, allowing for user input during or shortly after data acquisition.

FIG. 3 shows a schematic representation of a first image and a second image and operations performed for registering the images according to an exemplary embodiment of the present invention. The first image 301 is an image slice from a multi-dimensional data set, acquired, for example, by means of an MRI scanner system or an ultrasound imaging system. Image slice 301 visualizes a region of interest 303, defining, e.g., a lesion. After a certain time, a second image slice 302 is acquired and the region of interest defining the lesion is identified. Due to organ movement, the location of the lesion in image slice 302 may be shifted with respect to the location 303 of the lesion in image slice 301. Due to little or even no anatomical contrast, the identification of the object of interest (lesion) may not be possible. Therefore, according to an exemplary embodiment of the present invention, a whole set of possible positions of the object of interest in image slice 302 is identified, the set comprising region 304, region 305 and region 306. Furthermore, a translation is identified from the first region of interest 303 (see image slice 301) to the region of interest 304. This translation is translation 307. A further translation 308 is identified, referring to a translation of the region of interest 303 to the region of interest 305 and a third translation 309 is identified, referring to a translation of the region of interest 303 to the region of interest 306. Since the three regions of interest 304, 305, 306 are slightly shifted with respect to each other, translations 307, 308, 309 are slightly varied as well. These translations are now, according to an exemplary embodiment of the present invention, compared with each other, by deriving certain quality values for each of the three translations. After that, the “best” of the three translations is selected for registration of the two image slices 301, 302.

Although the present invention is described with reference to medical imaging, where the regions of interest relate to, for example, cancerous tissue, it should be noted that the present invention may also be applied to non-medical applications, for example, in material testing or quality control, where moving images of actual products are to be registered.

FIG. 4 shows a schematic representation of the operations performed for registering a time series of images according to an exemplary embodiment of the present invention.

Image 410 represents possible movements of a region of interest 303. According to the exemplary embodiment of the present invention depicted in FIG. 4, three different trajectories of translation vectors for a movement of the region of interest movement 303 are selected and compared with each other. The first translation vector describes the translation of the region of interest 303 to the location of 304 by translation 307 and then to location 402 by translation 407 and then to location 405 by translation 412. The second translation vector describes a translation of the region of interest 303 to location 305, then to location 403 and then to location 406 by respective translations 308, 408 and 413. A third translation vector describes the translation of the region of interest 303 to location 306, then to location 401 and then to location 404 by respective translations 309, 409 and 411. It should be noted that locations 304, 305 and 306 refer to a second image slice acquired at a second time, which is later than the first time at which the first image slice comprising location 303 is acquired. Furthermore, locations 401, 402 and 403 relate to a third image slice acquired at a third time later than the second time and locations 404, 405 and 406 relate to a fourth image slice acquired at a fourth (latest) time.

According to an aspect of the present invention, after determination of the three translation vector sequences linking region 303 with one of the regions 405, 404, or 406, the respective compartment vectors, each comprising compartment parameters, are determined on the basis of the three translation vector sequences and corresponding specific uptake values (which are measured in each image and for each region of interest) and a corresponding pharmacokinetic model or compartment model, which may be interactively selected by a user. On the basis of the compartment vectors, a quality determination is performed and one of the three translation vectors is selected, which relates to the “best” quality value derived from the quality determination. If, according to an exemplary embodiment of the present invention, the respective “best” quality value exceeds a preset threshold value, the corresponding translation vector is used for registration of the four images.

In other words: let R denote a region of interest enclosing a suspicious lesion, let I₁, . . . , Iₘ denote nuclear-medical images acquired at times t₁, . . . , tₘ and let Tₖ denote the translation of the region of interest R when progressing from image Iₖ to the next image Iₖ₊₁ in the time series. For every sequence of translations T₁=(T_{i,1}, . . . , T_{i,m-1}), estimates of the compartment parameters k_{i,1}, . . . , k_{i,m} are obtained and the quality of these estimates is determined with respect to some quality measure Q (with i=1, . . . ,r; r being the number of different sequences of translations or translation vectors). The time series is locally registered with respect to R for that particular sequence of translations Tⱼ that leads to the best quality of the estimates of compartment parameters k_{j,1}, . . . , k_{j,m}.

Possible choices for a quality measure Q are:
The statistical quality of the k-estimates, for example the statistical variance,
given a library of possible sets of k-values for the lesion and anatomy imaged, the distance in feature space from the closest matching set of k-values, or
the consistency of the k-estimates obtained from registering the image series forwards and backwards in time.

The method may be applied to any time series of two- or three-dimensional nuclear-medical image data sets, provided a pharmacokinetic model is available for the tracer used and the anatomical region imaged. Obtaining accurate and reproducible measures for tracer up-take is of crucial importance for determining the malignancy of suspicious lesions, the response of therapy and the early detection of recurring cancer. The application domain of this technique will rapidly grow with the rapid progress in the field of molecular imaging and the proliferation of specific tracers.

Advantageously, the present invention allows for a tracking of a region of interest through a time series of nuclear images while preserving shape and size of the lesion of interest. Furthermore, it may allow for a compensation of the patient or organ motion in order to obtain the best estimates possible for parameters characterizing tracer up-take. Thus, images with little or no anatomical contrast (as frequently encountered for highly specific tracers) may be successfully registered. Merely by analyzing a locally defined region of interest, the proposed method may allow for an increased registration speed.

Thus, the present invention allows for a global consistency of the estimates of the compartment parameters throughout the time series and therefore for an improved image registration, since a whole set of images (time series) together with a solid model describing the time dependency of tracer uptake are used for the registration (and not only, for example, two images and corresponding grey-value structures).

1. A method of registering a time series of images, the time series comprising at least a first image and a second image, the method comprising the steps of:
selecting a first region of interest in the first image;
selecting a second region of interest in the second image;
determining a first translation from the first region of interest to the second region of interest on the basis of a pharmacokinetic model;
registering the first image and the second image on the basis of the first translation;
wherein the first region of interest corresponds to the second region of interest.

2. The method according to claim 1, wherein the determination of the first translation comprises the steps of:
determining a first pharmacokinetic parameter on the basis of a second translation from the first region of interest to a third region of interest selected in the second image and on the basis of the pharmacokinetic model;
determining a second pharmacokinetic parameter on the basis of a third translation from the first region of interest to a fourth region of interest selected in the second image and on the basis of the pharmacokinetic model, wherein the third translation is a variation of the second translation and wherein the third and the fourth regions of interest correspond to the first region of interest;
performing a quality determination of the first compartment parameter and the second compartment parameter, resulting in a first quality value and a second quality value;
determining, which one of the first and second quality values is a better quality value;
selecting a translation from the second translation and the third translation; wherein the selected translation is the translation corresponding to the better quality value; and
wherein the selected translation is the first translation.

3. The method according to claim 2, wherein the quality determination is performed on the basis of at least one of:
a determination of a statistical parameter on the basis of at least one of the first pharmacokinetic parameter estimate and the second pharmacokinetic parameter estimate;
a library of pharmacokinetic parameters; and
a consistency of the first pharmacokinetic parameter estimate and the second pharmacokinetic parameter estimate.

4. The method according to claim 1, wherein at least one of the first and second regions of interest and the pharmacokinetic model are interactively selected from a predefined set of options.

5. The method according to claim 2, wherein the method is iteratively repeated until at least one of the first quality value and the second quality value exceeds a preset threshold value.

6. The method according to claim 1, wherein the method is applied in medical imaging to one of CT data sets, MRI data sets, PET data sets, SPECT data sets, and ultra sound imaging data sets.

7. An image processing device for registering a first image and a second image, the image processing device comprising: a memory for storing a multi-dimensional data set comprising the first image and the second image; an image
processor adapted for performing the following operation:
loading the multi-dimensional data set;
selecting a first region of interest in the first image;
selecting a second region of interest in the second image;
determining a first translation from the first region of interest to the second region of interest on the basis of a pharmacokinetic model; registering the first image and the second image on the basis of the first translation; wherein the first region of interest corresponds to the second region of interest.

8. A scanner system for registering a first image and a second image, the scanner system comprising:
a memory for storing a multi-dimensional data set comprising the first image and the second image;
an image processor adapted for performing a registration of the first image and the second image, wherein the image processor is adapted for performing the following operation:
loading the multi-dimensional data set;
selecting a first region of interest in the first image;
selecting a second region of interest in the second image;
determining a first translation from the first region of interest to the second region of interest on the basis of a pharmacokinetic model;
registering the first image and the second image on the basis of the first translation; wherein the first region of interest corresponds to the second region of interest.

9. The scanner system according to claim 8, wherein the scanner system is one of a CT scanner system, an MRI scanner system, a PET scanner system, a SPECT scanner system, and an ultrasound imaging system.

10. Computer program for registering a first image and a second image, wherein the computer program causes an image processor to perform the following operation when the computer program is executed on the image processor:
loading the multi-dimensional data set comprising the first image and the second image;
selecting a first region of interest in the first image;
selecting a second region of interest in the second image;
determining a first translation from the first region of interest to the second region of interest on the basis of a pharmacokinetic model;
registering the first image and the second image on the basis of the first translation;
wherein the first region of interest corresponds to the second region of interest.
